(19) United States
(12) Patent Application Publication
(10) Pub. No.: US 2011/0184315 A1
(43) Pub. Date: Jul. 28, 2011

(54) Multi-Loop Bridge-Type Apparatus for the Collection of Cells

(75) Inventors: Chih-Ping Chen, Taipei City (TW); Shu-Jen Chen, Taipei City (TW)

(73) Assignee: MACKAY MEMORIAL HOSPITAL, Taipei City (TW)

(21) Appl. No.: 12/787,994

(22) Filed: May 26, 2010

(30) Foreign Application Priority Data
Jan. 25, 2010 (TW) 099201495

(51) Int. Cl. A61B 10/00

(52) U.S. Cl. 600/573

(57) ABSTRACT

A multi-loop bridge-type apparatus for the collection of cells is provided. The multi-loop bridge-type apparatus includes a barrel, a plunger, a filter unit, and at least two external ducts. The barrel includes one open end. The filter unit is disposed inside an inner space of the barrel and partitions the inner space of the barrel into a first space and a second space. The open end is structured for connection to the first space. The plunger is coupled with the barrel and is adapted for executing a piston motion along the second space. Each external duct has two ends respectively communicating with the first space and the second space. Each external duct further includes a control valve for controlling the duct to be conducted or shut off.

### BACKGROUND OF THE INVENTION

1. Field of the Invention

The present invention relates generally to an apparatus for the collection of cells, and more particularly, to a multi-loop bridge-type apparatus for the collection of cells from amniotic fluid.

2. The Prior Arts

Amniotic fluid is the liquid contained by the amniotic sac and surrounding the fetus for nourishing and buffering the fetus. The amniotic fluid provides a space like a warm swimming pool allowing an embryo to grow up, excise, and adjust position. During the first 16 weeks after conception, most of the liquid is constituted of the liquids secreted from the skin of the fetus, and surfaces of the placenta and the umbilical cord. However, afterwards, the fetus starts to swallow these liquids, and meanwhile the kidney of the fetus starts to function so that the fetus starts to excrete urine to the amniotic sac. In such a way, amniotic fluid is continually being swallowed and “inhaled” and replaced through being “exhaled”, as well as being urinated by the fetus, thus constructing a circulation of amniotic fluid. As a result, the volume of the amniotic fluid drastically increases after the first 16 weeks, and at the same time the fetus obtain enough space for fast growing up. In the latest stage of gestation, the placenta has become much less active, and the blood provided to the fetus therefrom decrease, so that the urine excreted from the kidney of the fetus correspondingly decreases. The amount of the amniotic fluid also decreases until the birth of the baby.

Amniotic fluid contains cells peeled off from the skin, lung, or gastrointestinal tract of the fetus, and therefore is an important material for prenatal diagnosis. Generally, by detecting the ingredient variation of the amniotic fluid, doctors can learn the maturity of the fetus, and whether the fetus has any congenital defector or intrauterine infection. These cells can be bred with a tissue culture technique, and amniotic fluid sample is usually obtained by an amniocentesis operation conducted at the abdominal wall of the pregnant woman. However, the amniocentesis operation may possibly injure the fetus and/or the mother. Therefore, the clinical doctor must be very skillful in amniotic fluid analysis and amniocentesis operation, and the amniocentesis operation should be conducted about 16 weeks after the conception when the volume of the amniotic fluid reaches 200 ml.

In general, at about 15 to 16 weeks after the conception, the amniocentesis operation is allowed to be conducted, and at least 20 ml of amniotic fluid must be extracted for obtaining enough cells therefrom for cultivation. However, when the kidney of the is not in function yet, the total volume of the amniotic fluid is only about 100 ml to 150 ml, and therefore the 20 ml extracted amniotic fluid is really a large proportion.

Currently, a conventional apparatus typically used for collecting amniotic fluid during a prenatal diagnosis includes a puncture needle, and a 50 ml syringe. The conventional apparatus is used to extract about 20 ml amniotic fluid from the mother's body, and then the cells are separated from the extracted amniotic fluid. The separated cells are then cultivated for one to two weeks. Generally, after the cells are separated therefrom, the rest amniotic fluid would not be returned back to the mother's body. As such, the prenatal diagnosis conducted with the conventional apparatus consumes a large volume of the amniotic fluid, and is unfavorable to the fetus and the mother.

Further, there is a research proposed to refill the rest amniotic fluid back to the mother's body. However, the appliance required by the research is constituted of at least four independent components and is rather complicated. Those skilled in the art would have been very clear that more components used in the operation bring more risk of contaminating the extracted amniotic fluid sample. Contaminated sample would certainly fail the cells cultivation. Even further, when the contaminated amniotic fluid is refilled back to the mother's body, it may badly harm the health or even life of the mother and/or the fetus.

### SUMMARY OF THE INVENTION

Accordingly, an objective of the present invention is to provide a multi-loop bridge-type apparatus for the collection of cells. The multi-loop bridge-type cell apparatus can be used for extracting cells from amniotic fluid at an earlier stage of gestation for conducting an antenatal genetic diagnosis as earlier as possible. The multi-loop bridge-type apparatus of the present invention is adapted for extracting amniotic fluid from the uterus, and instantly processing the extracted amniotic fluid to obtain a first portion and a second portion. The first portion has a high concentration of cells from amniotic fluid and the second portion has amniotic fluid. The first portion is collected and the second portion is filled back to the uterus.

The present invention provides a multi-loop bridge-type apparatus for the collection of cells. The multi-loop bridge-type apparatus includes a barrel, a plunger, a filter unit, and at least two external ducts. The barrel having one open end. The filter unit is disposed inside an inner space of the barrel and partitions the inner space of the barrel into a first space and a second space. The open end is structured for connection to the first space. The plunger is coupled with the barrel and is adapted for executing a piston motion along the second space. Each external duct having two ends respectively communicates with the first space and the second space. Each external duct further includes a control valve for controlling the external duct to be conducted or shut off.

In accordance with the present invention, the multi-loop bridge-type apparatus can be used for extracting amniotic fluid from the uterus, and refilling most of the amniotic fluid back to the uterus, so that the total amount of the amniotic fluid won't lose much due to the sampling operation. In such a way, the cells from amniotic fluid can be sampled without extracting much amniotic fluid. Further, the multi-loop bridge-type apparatus includes multiple loops (i.e. external ducts) for extracting the amniotic fluid, so that the resistance is small, and therefore the apparatus can be used for repetitive operation. Furthermore, the barrel is integrally formed, and according to an embodiment of the present invention, the external ducts are also integrally formed with the barrel. Therefore, additional contamination can be avoided.

### BRIEF DESCRIPTION OF THE DRAWINGS

The present invention will be apparent to those skilled in the art by reading the following detailed description of preferred embodiments thereof, with reference to the attached drawings, in which:

FIG. 1 is a cross-sectional view of a multi-loop bridge-type apparatus according to an embodiment of the present invention;

FIG. 2 illustrates an operation of using the multi-loop bridge-type apparatus to extract the amniotic fluid;

FIG. 3 illustrates an operation of using the multi-loop bridge-type apparatus to separate the cells from the amniotic fluid; and

FIG. 4 illustrates an operation of using the multi-loop bridge-type apparatus to collect the separated cells from amniotic fluid.

### DETAILED DESCRIPTION OF THE PREFERRED EMBODIMENT

The accompanying drawings are included to provide a further understanding of the present invention, and are incorporated in and constitute a part of this specification. The drawing illustrates embodiments of the present invention and, together with the description, serves to explain the principles of the present invention.

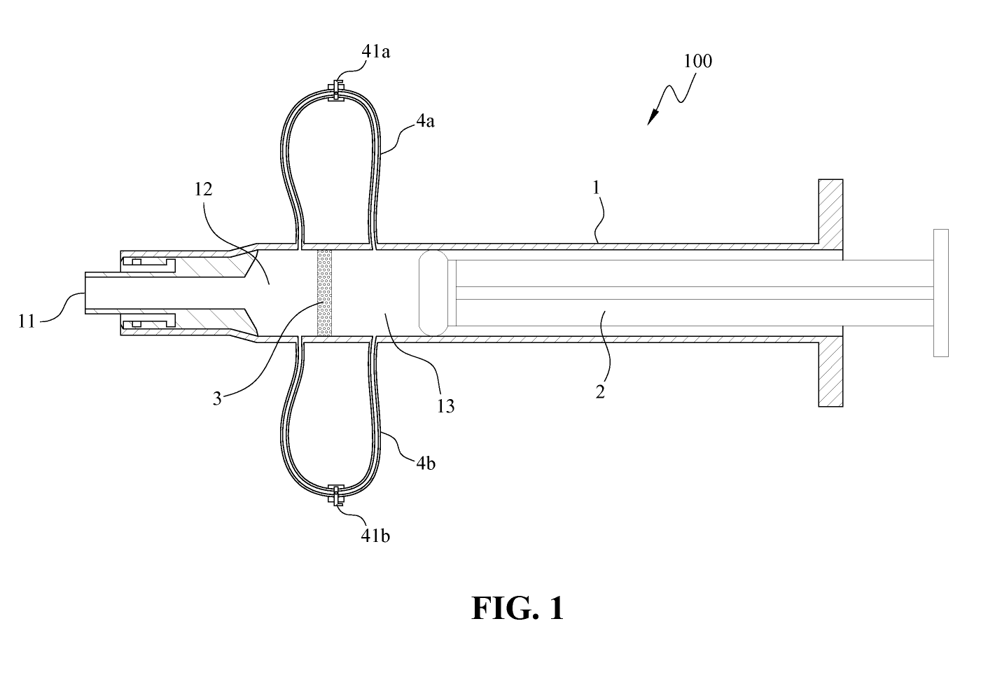

FIG. 1 is a cross-sectional view of a multi-loop bridge-type apparatus according to an embodiment of the present invention. Referring to FIG. 1, there is shown a multi-loop bridge-type apparatus 100. The multi-loop bridge-type apparatus 100 comprises a barrel 1, a plunger 2, a filter unit 3, and two external ducts 4a, 4b.

The barrel 1 has one open end 11 positioned at a fore-end of the barrel 1. The filter unit 3 is disposed inside an inner space of the barrel 1 and partitions the inner space of the barrel 1 into a first space 12 and a second space 13. The open end 11 is structured for connection to the first space 12. The plunger 2 is coupled with the barrel 1 and is adapted for executing a piston motion along the second space 13. When the plunger 2 is drawn outwardly from the second space 13, a sample is inhaled via the open end 11 into the first space 12, and when the plunger 2 is push inwardly to the second space 13, the sample contained in the first space 12 is exhaled via the open end 11. According to a preferred embodiment of the present invention, a plurality of pores of the filter unit 3 has a diameter of 0.4 μm for restricting cells greater than the diameter from passing therethrough.

According to an embodiment of the present invention, the external ducts 4a, 4b are jointly connected to the barrel 1, while according to another embodiment of the present invention, the external ducts 4a, 4b are integrally formed together with the barrel 1. The barrel 1 is integrally formed and thus the multi-loop bridge-type apparatus 100 can be conveniently maintained sterilized during the operation. Preferably, the external ducts 4a and 4b are similar to each other, and the external duct 4a is taken as an example for illustration. The external duct 4a has two ends respectively communicating with the first space 12 and the second space 13. The external duct 4a further includes a control valve 41a for controlling the external duct 4a to be conducted or shut off. Similarly, the external duct 4b also has two ends respectively communicating with the first space 12 and the second space 13. The external duct 4b also further includes a control valve 41b for controlling the external duct 4b to be conducted or shut off. According to an embodiment of the present invention, the barrel 1, and the external ducts 4a, 4b are preferred but not restricted to be made of PVC material. The barrel 1 has a capacity of about 25 ml. A diameter of the external ducts 4a, 4b is preferred but not restricted to be 4 mm.

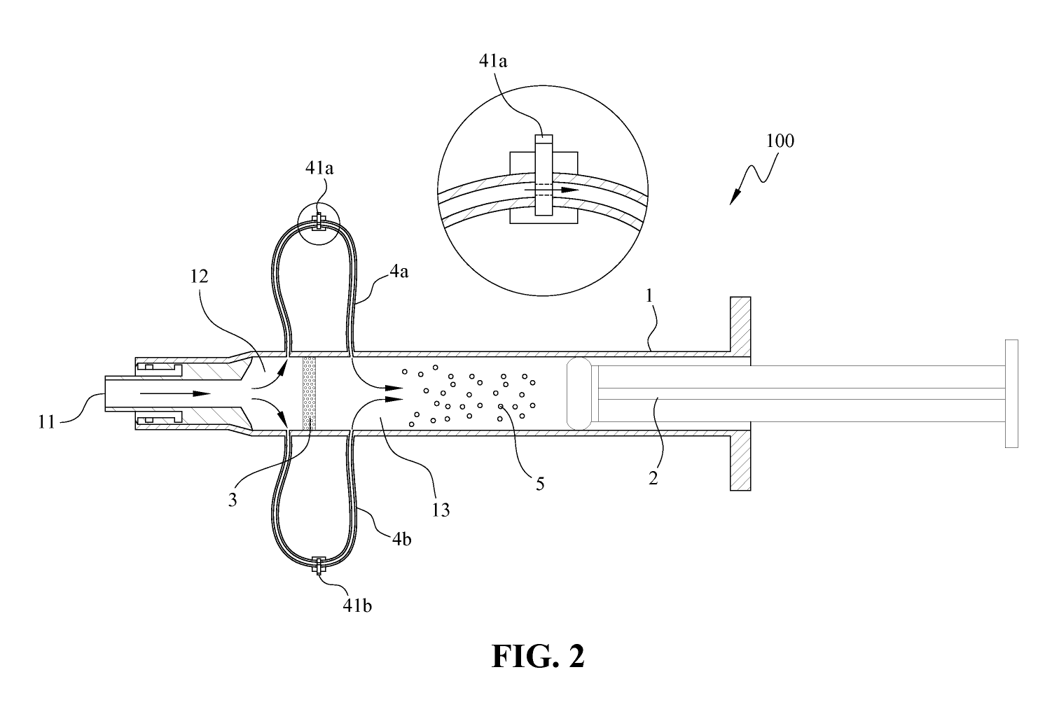

FIG. 2 illustrates an operation of using the multi-loop bridge-type apparatus to extract the amniotic fluid. Referring to FIG. 2, in operation, at first, the plunger 2 is drawn outwardly from the second space 13 to extract the amniotic fluid containing cells 5 from amniotic fluid via the open end 11 of the barrel 1 into the first space 12. The cells 5 from amniotic fluid are greater than the size of the pores of the filter unit 3, and are restricted from passing therethrough toward the second space 13, so that a local jam and a pressure difference between both sides of the filter unit 3 instantly occur. In this case, the control valves 41a and 41b are conducted, and thus the amniotic fluid together with the cells 5 from amniotic fluid flows from the first space 12 through the external ducts 4a and 4b into the second space 13. Accordingly, most of the cells 5 from amniotic fluid are now transported into the second space 13.

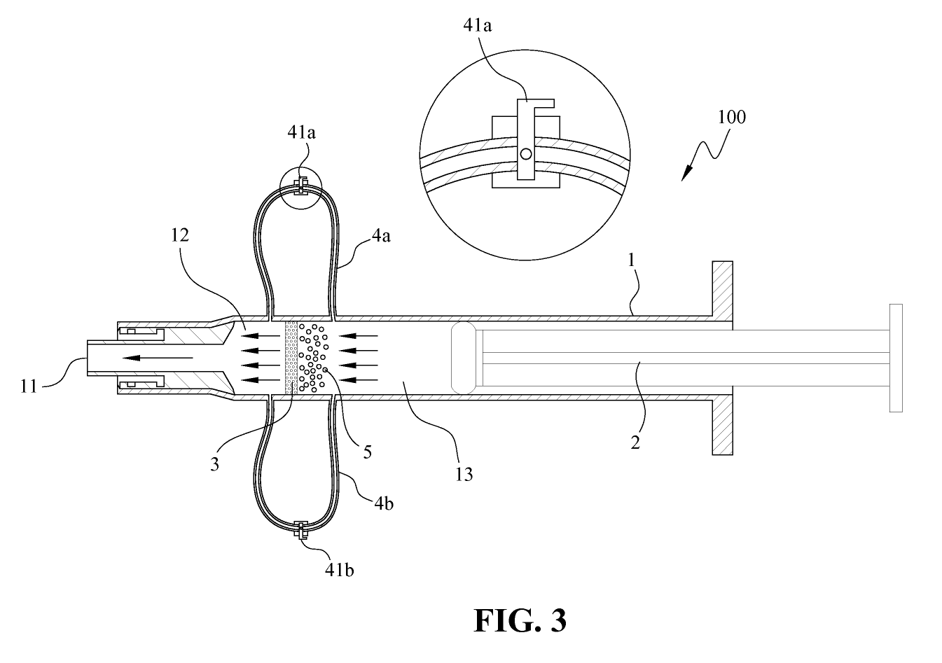

FIG. 3 illustrates an operation of using the multi-loop bridge-type apparatus to separate the cells from the amniotic fluid. After transporting most of the cells 5 from amniotic fluid into the second space 13, the control valves 41a and 41b are switched to be shut off, and then the plunger 2 is pushed inwardly toward the second space 13. In that way, the amniotic fluid contained in the second space 13 is pushed to pass through the filter unit 3 into the first space 12 and returned back to the mother's uterus, while most of the cells 5 from amniotic fluid are left in the second space 13.

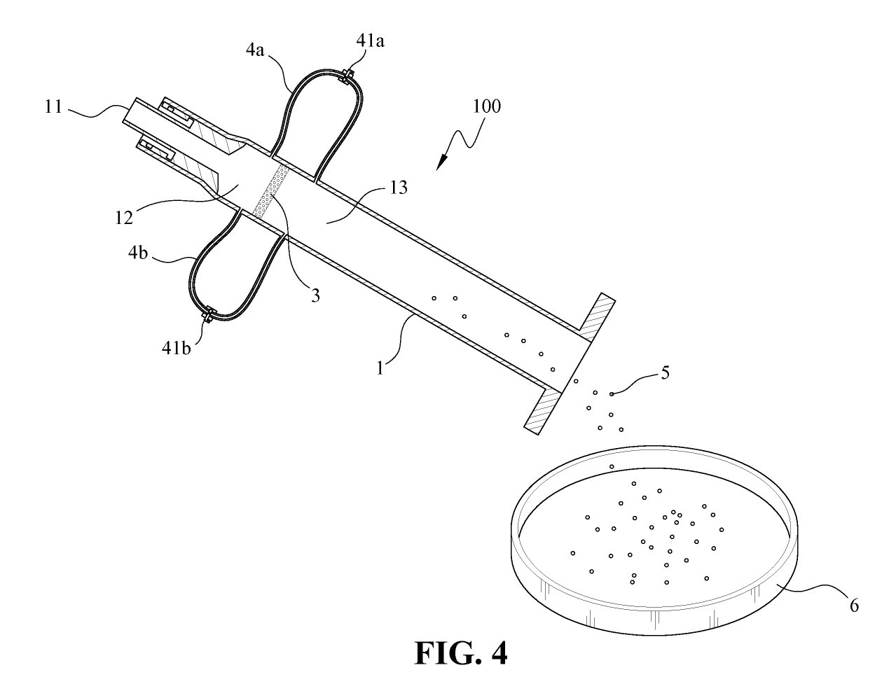

FIG. 4 illustrates an operation of using the multi-loop bridge-type apparatus to collect the separated amniotic fluid cell. Referring to FIG. 4, after returning back the amniotic fluid, the cells 5 from amniotic fluid left in the second space 13 of the barrel 1 are then removed therefrom and contained in a culture dish 6 for prenatal diagnosis.

It should be further noted, although two external ducts 4a, 4b are exemplified for illustrating the present invention, more external duct(s) could be adaptively employed in accordance with the spirit of the present invention.

Although the present invention has been described with reference to the preferred embodiments thereof, it is apparent to those skilled in the art that a variety of modifications and changes may be made without departing from the scope of the present invention which is intended to be defined by the appended claims.

What is claimed is:

1. A multi-loop bridge-type apparatus for collecting cells, comprising:
a barrel having one open end;
a filter unit disposed inside an inner space of the barrel and partitioning the inner space of the barrel into a first space and a second space, and the open end structured for connection to the first space;
a plunger coupled with the barrel and adapted for executing a piston motion along the second space; and
at least two external ducts, wherein each external duct having two ends respectively communicates with the first space and the second space, and each external duct further includes a control valve for controlling the external duct to be conducted or shut off.

2. The apparatus as claimed in claim 1, wherein the barrel is integrally formed.

3. The apparatus as claimed in claim 1, wherein the external ducts are integrally formed with the barrel.

4. The apparatus as claimed in claim 1, wherein the barrel is made of a PVC material.

5. The apparatus as claimed in claim 1, wherein the barrel has a capacity of 25 ml.

6. The apparatus as claimed in claim 1, wherein the external ducts are made of PVC materials.

7. The apparatus as claimed in claim 1, wherein each external duct has a diameter of 4 mm.

8. The apparatus as claimed in claim 1, wherein a plurality of pores of the filter unit has a diameter of 0.4 μm.

9. The apparatus as claimed in claim 1, wherein the cells are from an amniotic fluid.
